(19) United States
(12) Patent Application Publication
(10) Pub. No.: US 2002/0019372 A1
(43) Pub. Date: Feb. 14, 2002

(54) Use of ascorbic acid and salts of ascorbic acid to promote cell repair and regeneration after injury

(76) Inventors: Ricky Gene Schnellmann, Mount Pleasant, SC (US); Grazyna Nowak, Maumelle, AR (US)

Correspondence Address: Benjamin Aaron Adler, ADLER & ASSOCIATES, 8011 Candle Lane, Houston, TX, 77071, US

(21) Appl. No.: 09/899,704

(22) Filed: Jul. 5, 2001

Related U.S. Application Data
(60) Provisional application No. 60/215,960, filed on Jul. 5, 2000.
(60) Provisional application No. 60/212,224, filed on Jun. 16, 2000.

(51) Int. Cl. A61K 31/665; A61K 31/375

(52) U.S. Cl. 514/99; 514/474

(57) ABSTRACT

Ascorbic acid is a known vitamin and antioxidant. The present invention demonstrated that ascorbic acid is a strong promoter of cell repair and regeneration after injury. The mechanism by which ascorbic acid produces this effect is not through its known antioxidant properties. The present invention thus provides a new medical use for ascorbic acid in that ascorbic acid or its salts can be used as a drug following injury to promote cell repair and regeneration, and ultimately return of normal organ function. In addition, ascorbic acid or its salts could be given prior to a planned injury (e.g. surgery) to enhance cell repair and regeneration.

### CROSS-REFERENCE TO RELATED APPLICATION

This non-provisional patent application claims benefit of provisional patent application U.S. Serial No. 60/212,224 filed Jun. 15, 2000, now abandoned.

### FEDERAL FUNDING LEGEND

This invention was produced in part using funds obtained through a grant from the National Institutes of Health. Consequently, the federal government has certain rights in this invention.

### BACKGROUND OF THE INVENTION

1. Field of the Invention

The present invention relates generally to the field of cellular injury. More specifically, the present invention relates to the use of ascorbic acid and its salts in promoting recovery of cellular functions following cellular injury.

2. Description of the Related Art

Acute renal failure (ARF) is a condition of reduced renal function caused by an acute insult including ischemia and nephrotoxic compounds. Acute renal failure caused by toxicant-induced injury is often associated with injury and death of renal epithelial cells (Anderson and Schrier, 1997; Goldstein and Schnellmann, 1995). However, acute renal failure can also occur in the absence of visible tubular damage (Toback, 1992; Goldstein and Schnellmann, 1995). Numerous toxicants can cause renal dysfunction through their ability to induce sublethal injury to renal cells that results in decreased normal cellular functions without producing cell death and loss.

Renal proximal tubular cells (RPTC) play a major role in the reabsorption of ions, water, glucose, and solutes from the glomerular filtrate. Renal proximal tubular cells are the primary target of many toxicants due to their active transport functions and selective accumulation of xenobiotics. The most common alterations in renal proximal tubular cells caused by injury are: 1) loss and/or internalization of the brush border membrane microvilli, 2) mitochondrial dysfunction followed by ATP depletion and reduced metabolic functions, 3) decreased Na⁺-K⁺-ATPase activity, 4) loss of polarity of the plasma membrane, 5) altered ion homeostasis, and 6) altered transepithelial transport of ions and solutes followed by an impairment of renal proximal tubular cells reabsorptive functions (Venkatachalam et al., 1981; Molitoris et al., 1989; Meister et al., 1989; Molitoris, 1991; Mohrmann et al., 1993; Monteil et al., 1993; Kribben et al., 1994; Alejandro et al., 1995; Molck and Friis, 1997; Weinberg et al., 1997).

The Na⁺-K⁺-ATPase is responsible for the transmembrane movement of Na⁺ and K⁺ and mediates Na⁺ reabsorption by renal proximal tubular cells. The Na⁺-K⁺-ATPase is localized on the basolateral membrane where it forms a metabolically stable, detergent-insoluble complex with cytoskeletal proteins such as actin, fodrin, and ankyrin (Molitoris, 1991). Following ischemic injury, Na⁺-K⁺-ATPase polarity is lost due to redistribution of this protein, ankyrin and fodrin from the basolateral to the apical membrane (Spiegel et al., 1989; Molitoris, 1991). Other forms of cell injury that lead to the depletion of intracellular ATP also are accompanied by the dissociation of the Na⁺-K⁺-ATPase from the cytoskeleton and loss of the Na⁺-K⁺-ATPase function, resulting in decreased renal Na⁺ reabsorption (Molitoris, 1991). Injured renal proximal tubular cells are unable to restore Na⁺ reabsorption until the re-establishment of Na⁺-K⁺-ATPase localization on the basolateral membrane has occurred (Molitoris, 1991).

Halogenated hydrocarbons represent a large group of chemicals that produce toxicity after their biotransformation to nephrotoxic cysteine S-conjugates (Elfarra et al., 1986; Dekant et al., 1994). Dichlorovinyl-L-cysteine (DCVC) is a model halocarbon nephrotoxicant that is selective for renal proximal tubular cells and produces renal proximal tubular cell necrosis and acute renal failure (Stevens et al., 1986; Van der Water et al., 1994). In renal proximal tubular cells, dichlorovinyl-L-cysteine is transformed to a thiol-containing reactive metabolite that produces nephrotoxicity through covalent binding to target cellular molecules and inhibition of renal proximal tubular cell functions (Stevens et al., 1986; Chen et al., 1994, Groves et al., 1993). Furthermore, oxidative stress also was implicated in the mechanism of dichlorovinyl-L-cysteine-induced injury in renal proximal tubular cell (Groves et al., 1991). Acute exposure of renal proximal tubular cells to dichlorovinyl-L-cysteine results in the loss of Ca²⁺ homeostasis, mitochondrial dysfunction and ATP depletion, lipid peroxidation, DNA damage, loss of brush border enzymes, decreased Na⁺-K⁺-ATPase activity and active Na⁺ transport, and inhibition of renal proximal tubular cell transport functions (Lash and Anders, 1987; Groves et al., 1991; Groves et al., 1993; Chen et al., 1994; Lash, 1994; Van der Water et al., 1994; Nowak et al., 1999).

Renal proximal tubular cell have the capacity for restoring their structure and functions after nonlethal injury induced by toxicants and ischemia/reperfusion injury. The return of renal proximal tubular cell physiological functions is critical for the restoration of normal renal function (Toback, 1992; Toback et. al., 1993). Using an in vitro model of primary cultures of rabbit renal proximal tubular cells grown in improved culture conditions, it has been shown that renal proximal tubular cells proliferate and recover physiological functions following sublethal injury induced by the oxidant t-butylhydroperoxide (Nowak et al., 1998). In contrast, dichlorovinyl-L-cysteine-induced sublethal injury decreases renal proximal tubular cell mitochondrial function, Na⁺-K⁺-ATPase activity, active Na⁺ transport, and Na⁺-dependent glucose uptake but is not followed by the repair of these functions (Nowak et al., 1999). The mechanisms responsible for the inability of renal proximal tubular cell to repair their functions following dichlorovinyl-L-cysteine exposure are not known.

Thus, the prior art is deficient in an effective mean of restoring the function of renal proximal tubular cells after exposure to halocarbon nephrotoxicant. The present invention fulfills this long-standing need and desire in the art.

### SUMMARY OF THE INVENTION

It has been shown previously that L-ascorbic acid phosphate (AscP) promoted the growth, mitochondrial and transport functions in primary cultures of renal proximal tubular cells (RPTC) (Nowak and Schnellmann, 1996). Furthermore, L-ascorbic acid phosphate stimulated regeneration of the renal proximal tubular cells monolayer following oxidant-induced injury by stimulation of proliferation and migration/spreading (Nowak and Schnellmann, 1997). However, it is not known whether L-ascorbic acid phosphate promotes recovery of renal proximal tubular cells functions following injury induced by halocarbon nephrotoxicant such as dichlorovinyl-L-cysteine.

The present study was designed to address this issue, and results from the present invention indicate that: 1) proliferation, mitochondrial function, Na⁺-K⁺-ATPase protein level and activity, and active Na⁺-transport do not recover in dichlorovinyl-L-cysteine-injured renal proximal tubular cells cultured in the presence of physiological concentrations of L-ascorbic acid phosphate; 2) pharmacological concentrations of L-ascorbic acid phosphate promote proliferation and repair of mitochondrial function, recovery of Na⁺-K⁺-ATPase protein level and activity, and return of active Na⁺ transport in dichlorovinyl-L-cysteine-injured renal proximal tubular cells; and 3) stimulation of proliferation and recovery of mitochondrial function and active Na⁺ transport in renal proximal tubular cells by pharmacological concentrations of L-ascorbic acid phosphate is not due to protective effects of L-ascorbic acid phosphate against dichlorovinyl-L-cysteine-induced cell death and/or decreases in mitochondrial function, Na⁺-K⁺-ATPase activity, and active Na⁺ transport. These data also suggest that the beneficial effects of pharmacological concentrations of ascorbic acid in the kidney are not limited to antioxidant action of this molecule and that ascorbic acid may be an important tool in promoting recovery of renal functions following toxicant-induced injury.

It is an object of the present invention to use ascorbic acid and its salts to promote cell repair and regeneration.

In one embodiment of the present invention, there is provided a method of recovering cellular functions in cells following injury by contacting the cells with pharmacological concentrations of ascorbic acid or its salts. Preferably, L-ascorbic acid phosphate is used to promote proliferation and repair of mitochondrial function, recovery of Na⁺-K⁺-ATPase protein level and activity, and return of active Na⁺ transport in halogenated hydrocarbons-injured renal proximal tubular cells.

In another embodiment of the present invention, there is provided a pharmaceutical composition, comprising ascorbic acid or its salts and a pharmaceutically acceptable carrier. Preferably, the pharmaceutical composition is useful for ophthalmic applications or topical applications.

In yet another embodiment of the present invention, there is provided a method of recovering cellular functions following injury in an individual using a pharmaceutical composition of ascorbic acid. Preferably, such injury are halogenated hydrocarbon-induced nephrotoxicity, ischemia- and drug-induced acute renal failure, glomerulonephritis, acute injury to the eye, eye diseases associated with the over production of collagen (conjunctivitis, diabetes mellitus), eye disease associated with the under production of collagen (alkali burns, rheumatoid arthritis), and skin abrasions, cuts, and burns. More preferably, such treatment is used to promote proliferation and repair of mitochondrial function, recovery of Na⁺-K⁺-ATPase protein level and activity, and return of active Na⁺ transport.

In yet another embodiment of the present invention, there is provided a product for delivery of a therapeutically effective amount of ascorbic acid comprising: (A) a strip comprising: (i) a flexible substrate sheet; and (ii) a therapeutically effective amount of ascorbic acid deposited onto said substrate sheet.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the effects of 0.05 and 0.5 mM L-ascorbic acid phosphate on recovery of renal proximal tubular cells monolayer DNA content following dichlorovinyl-L-cysteine (0.2 mM) exposure. Renal proximal tubular cells were cultured in the presence of 0.05 and 0.5 mM L-ascorbic acid phosphate prior to and following dichlorovinyl-L-cysteine exposure. Data are means ±SE; n=3 separate experiments. Values with different letters on a given day are significantly different (P<0.05) from each other.

FIG. 2 shows the effects of 0.05 and 0.5 mM L-ascorbic acid phosphate on recovery of renal proximal tubular cells basal oxygen consumption (QO₂) following dichlorovinyl-L-cysteine (0.2 mM) exposure. Renal proximal tubular cells were cultured in the presence of 0.05 and 0.5 mM L-ascorbic acid phosphate prior to and following dichlorovinyl-L-cysteine exposure. Data are means ±SE; n=6 separate experiments. Values with different letters on a given day are significantly different (P<0.05) from each other.

FIG. 3 shows the effects of 0.05 and 0.5 mM L-ascorbic acid phosphate on recovery of renal proximal tubular cells ouabain-sensitive oxygen consumption (QO₂) following dichlorovinyl-L-cysteine (0.2 mM) exposure. Renal proximal tubular cells were cultured in the presence of 0.05 and 0.5 mM L-ascorbic acid phosphate prior to and following dichlorovinyl-L-cysteine exposure. Data are means ±SE; n=5 separate experiments. Values with different letters on a given day are significantly different (P<0.05) from each other.

FIG. 4 shows the effects of 0.05 and 0.5 mM L-ascorbic acid phosphate on recovery of renal proximal tubular cells Na⁺-K⁺-ATPase activity following dichlorovinyl-L-cysteine (0.2 mM) exposure. Renal proximal tubular cells were cultured in the presence of 0.05 and 0.5 mM L-ascorbic acid phosphate prior to and following dichlorovinyl-L-cysteine exposure. Data are means ±SE; n=4 separate experiments. Values with different letters on a given day are significantly different (P<0.05) from each other.

FIG. 5 shows the confocal laser scanning images of α1 subunit of Na⁺-K⁺-ATPase on the apical (A, C, and E) and basolateral (B, D, and F) domain of control (A and B) and sublethally-injured renal proximal tubular cells on day 1 (C and D) and day 4 (E and F) following dichlorovinyl-L-cysteine (0.2 mM) exposure. Renal proximal tubular cells were cultured in the presence of 0.05 mM AscP prior to and following dichlorovinyl-L-cysteine exposure (magnification 800×).

FIG. 6 shows the confocal laser scanning images of α1 subunit of Na⁺-K⁺-ATPase on the apical (A, C, and E) and basolateral (B, D, and F) domain of control (A and B) and sublethally-injured renal proximal tubular cells on day 1 (C and D) and day 4 (E and F) following dichlorovinyl-L-cysteine (0.2 mM) exposure. Renal proximal tubular cells were cultured in the presence of 0.5 mM AscP prior to and following dichlorovinyl-L-cysteine exposure (magnification 800×).

### DETAILED DESCRIPTION OF THE INVENTION

It has been shown that renal proximal tubular cells recover cellular functions following sublethal injury induced by the oxidant t-butylhydroperoxide but not by the nephrotoxic cysteine conjugate dichlorovinyl-L-cysteine. The present study investigated whether L-ascorbic acid phosphate promotes recovery of renal proximal tubular cells functions following dichlorovinyl-L-cysteine-induced injury. Dichlorovinyl-L-cysteine exposure (0.2 mM; 100 min) resulted in 60% renal proximal tubular cells death and loss from the monolayer at 24 hr independent of physiological (0.05 mM) or pharmacological (0.5 mM) AscP concentrations. Likewise, the dichlorovinyl-L-cysteine-induced decrease in mitochondrial function (54%), active Na⁺ transport (66%), and Na⁺-K⁺-ATPase activity (77%) was independent of the AscP concentration. Analysis of Na⁺-K⁺-ATPase protein expression and distribution in the plasma membrane using immunocytochemistry and confocal laser scanning microscopy revealed the loss of Na⁺-K⁺-ATPase protein from the basolateral membrane of renal proximal tubular cells treated with dichlorovinyl-L-cysteine. DCVC-injured renal proximal tubular cells cultured in the presence of 0.05 mM AscP did not proliferate nor recover their physiological functions over time. In contrast, renal proximal tubular cells cultured in the presence of 0.5 mM AscP proliferated, recovered all examined physiological functions and the basolateral membrane expression of Na⁺-K⁺-ATPase by day 4 following dichlorovinyl-L-cysteine injury. These results demonstrate that pharmacological concentrations of AscP do not prevent toxicant-induced cell injury and death but promote complete recovery of mitochondrial function, active Na⁺-transport, and proliferation following toxicant-induced injury. These data also suggest that the recovery of renal proximal tubular cells functions following toxicant exposure produced by AscP is not due to an antioxidant effect.

It is an object of the present invention to use ascorbic acid and its salts to promote cell repair and regeneration. It is specifically contemplated that pharmaceutical compositions may be prepared using a pharmacological concentration of ascorbic acid or its salts disclosed in the present invention. It is not intended that the present invention be limited by the particular nature of the therapeutic preparation, so long as the preparation comprises ascorbic acid or its salts. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual hosts. A person having ordinary skill in this art would readily be able to determine, without undue experimentation, the appropriate dosages and routes of administration of ascorbic acid of the present invention.

Ascorbic acid, also known by its common name of Vitamin C, is a very unstable substance. Although readily soluble in water, rapid oxidation occurs in aqueous media. Solubility of ascorbic acid has been reported to be relatively poor in nonaqueous media, thereby preventing an anhydrous system from achieving a significant level of active concentration. Derivatives have been produced with greater stability than the parent component. See U.S. Pat. No. 5,137,723 (Yamamoto et al.) and U.S. Pat. No. 5,078,989 (Ando et al.) A two-pack approach has been developed where Vitamin C powder and other ingredients are separately packaged in different containers with mixing just prior to use. See U.S. Pat. No. 4,818,521 (Tamabuchi). Water compatible alcohols such as propylene glycol, polypropylene glycol and glycerol have been used as co-carriers alongside water to improve stability. See U.S. Pat. No. 4,983,382 (Wilmott and Znaiden).

In one embodiment of the present invention, there is provided a method of recovering cellular functions in cells following injury by contacting the cells with pharmacological concentrations of ascorbic acid or its salts. Preferably, L-ascorbic acid phosphate is used to promote proliferation and repair of mitochondrial function, recovery of Na⁺-K⁺-ATPase protein level and activity, and return of active Na⁺ transport in halogenated hydrocarbons-injured renal proximal tubular cells.

In another embodiment of the present invention, there is provided a pharmaceutical composition, comprising ascorbic acid or its salts and a pharmaceutically acceptable carrier. Such compositions are typically prepared as liquid solutions or suspensions, or in solid forms. The compositions are also prepared as injectables, either as liquid solutions or suspensions; solid forms suitable for solution in, or suspension in, liquid prior to injection may also be prepared. When used in vivo for therapy, the ascorbic acid of the present invention is administered to the patient or an animal in therapeutically effective amounts, i.e., amounts that enhance cell repair and recovery of cell functions after injury. It will normally be administered parenterally, preferably intravenously, but other routes of administration will be used as appropriate. The dose and dosage regimen will depend upon the nature of the injury and diseases. The schedule will be continued to optimize effectiveness while balanced against negative effects of treatment. See Remington's Pharmaceutical Science, 17th Ed. (1990) Mark Publishing Co., Easton, Penn.; and Goodman and Gilman's: The Pharmacological Basis of Therapeutics, 8th Ed (1990) Pergamon Press; which are incorporated herein by reference.

In another embodiment, the present invention contemplates an ophthalmic composition of ascorbic acid or its salts in the form of aqueous eye drops, liposomes, microspheres, proteins, collagen, or soft contact lenses.

In still yet another embodiment, the present invention contemplates topical administration of ascorbic acid or its salts using solid supports (such as dressings and other matrices) and medicinal formulations (such as creams, lotions, ointments and in some cases, suppositories). In one embodiment, the solid support comprises a dressing. In still another embodiment, the solid support comprises a band-aid. The term “solid support” refers broadly to any support, including, but not limited to, microcarrier beads, gels, Band-Aids.™ and dressings. The term “dressing” refers broadly to any material applied to a wound for protection, absorbance, drainage, etc. Thus, adsorbent and absorbent materials are specifically contemplated as a solid support. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer) [Kannon and Garrett, Dermatol. Surg. 21:583-590 (1995); Davies, Burns 10:94 (1983)]. The present invention specifically contemplates the use of dressings impregnated with ascorbic acid of the present invention. The term “Band-Aid.” is meant to indicate a relatively small adhesive strip comprising an adsorbent pad (such as a gauze pad) for covering minor wounds.

In yet another embodiment of the present invention, there is provided a method of recovering cellular functions following injury in an individual using one of the above pharmaceutical compositions of ascorbic acid or its salts. Preferably, such injury are halogenated hydrocarbon-induced nephrotoxicity, ischemia- and drug-induced acute renal failure, glomerulonephritis, acute injury to the eye, eye diseases associated with the over production of collagen (conjunctivitis, diabetes mellitus), eye disease associated with the under production of collagen (alkali burns, rheumatoid arthritis), and skin abrasions, cuts, and burns. More preferably, such treatment is used to promote proliferation and repair of mitochondrial function, recovery of Na⁺-K⁺-ATPase protein level and activity, and return of active Na⁺ transport.

The following examples are given for the purpose of illustrating various embodiments of the invention and are not meant to limit the present invention in any fashion.

### EXAMPLE 1

Reagents

Female New Zealand White rabbits (1.5-2.0 kg) were purchased from Myrtle's Rabbitry (Thompson Station, Tenn.). S-(1,2dichlorovinyl)-L-cysteine (DCV) was a generous gift from Dr. T. W. Petry (Pharmacia Upjohn, Kalamazoo, Mich.) and was synthesized according to the method of Moore and Green (1988). Sodium dodecyl sulfate (SDS) was obtained from Bio-Rad (Hercules, Calif.). L-Ascorbic acid-2-phosphate magnesium salt and cell culture media were obtained from Wako BioProducts (Richmond, Va.) and Life Technologies (Grand Island, N.Y.), respectively. Anti-rabbit Na⁺ /K⁺-ATPase subunit α1 monoclonal antibody was supplied by Upstate Biotechnology (Lake Placid, N.Y.). FITC-conjugated goat anti-mouse IgG was purchased from Chemicon (Temecula, Calif.). The sources of the other reagents have been described previously (Nowak and Schnellmann, 1996; Nowak et al., 1998; Nowak et al., 1999).

### EXAMPLE 2

Isolation of Proximal Tubules and Culture Conditions

Rabbit renal proximal tubules were isolated by iron oxide perfusion method and grown in 35 mm culture dishes in improved conditions as described previously (Nowak and Schnellmann, 1996). The purity of the renal proximal tubular S₁ and S₂ segments isolated by this method is approximately 96%. The culture medium was a 50:50 mixture of Dulbecco's modified Eagle's essential medium (DMEM) and Ham's F-12 nutrient mix without phenol red, pyruvate, and glucose, supplemented with 15 mM NaHCO₃, 15 mM N-2hydroxyethylpiperazine-N′-2-ethanesulfonic acid, and 6 mM lactate (pH7.4, 295 mosmol/kg). Human transferrin (5 μg/ml), selenium (5 ng/ml), hydrocortisone (50 nM), bovine insulin (10 nM), and L-ascorbic acid-2-phosphate (0.05 mM or 0.5 mM) were added to the medium immediately before daily media change (2 ml/ dish).

### EXAMPLE 3

Toxicant Treatment of Renal Proximal Tubular Cells Monolayer

Renal proximal tubular cells monolayers reached confluence within 5 days and were treated with dichlorovinyl-L-cysteine (0.2 mM, 100 min) on day 6 of culture. Following dichlorovinyl-L-cysteine exposure, the remaining monolayer was washed with fresh medium and cultured for 4 days. Samples of renal proximal tubular cells were taken at various time points after dichlorovinyl-L-cysteine exposure for measurements of cellular functions. Prior to measurement of any functions, renal proximal tubular cells were washed with ice cold phosphate buffered saline (pH 7.4) or 37° C. culture media (for measurement of oxygen consumption, QO₂) to remove non-viable cells.

### EXAMPLE 4

Oxygen Consumption

Washed renal proximal tubular cells monolayers were gently detached from the dishes with a rubber policeman, suspended in 37° C. culture medium and transferred to the oxygen consumption (QO₂) measurement chamber. QO₂ was measured polarographically using Clark type electrode as described previously (Nowak and Schnellmann, 1996; Nowak et al., 1998; Nowak et al., 1999). Ouabain-insensitive QO₂ was measured in the presence of 0.1 mM ouabain and was calculated as a difference between basal and ouabain-insensitive QO₂.

### EXAMPLE 5

Measurement of Na⁺-K⁺-ATPase Activity

Na⁺-K⁺-ATPase activity was determined in cellular lysates by measuring the difference between total ATPase activity and ouabain-insensitive ATPase activity using the method of Schwartz and Evan (1984). Cellular lysates were prepared as described by Forbush (1983). Briefly, 0.1-0.5 mg of renal proximal tubular cells protein was added to 0.1 ml of 25 mM imidazole buffer (pH 7.0) containing 0.065% SDS and 1% bovine serum albumin (BSA). Following incubation for 10 min at 22° C., 0.6 ml of 0.3% BSA in 25 mM imidazole buffer was added to lower the SDS concentration and 0.05 ml aliquots used for measurement of Na⁺-K⁺-ATPase activity.

### EXAMPLE 6

Assessment of Renal Proximal Tubular Cells Proliferation

Monolayer DNA content was used as a marker of renal proximal tubular cells proliferation. Monolayers were solubilized in 0.05 M Tris-HCl (pH 7.4) containing 0.15 M NaCl and 0.05% Triton X-100 and DNA determined in cell lysates by the method of Labarca and Paigen (1980) as described previously (Nowak and Schnellmann, 1996). Protein was measured by the method of Lowry et al. (1951).

### EXAMPLE 7

Immunocytochemical Localization of Na⁺-K⁺-ATPase

At various time points following dichlorovinyl-L-cysteine exposure, control and DCVC-treated renal proximal tubular cells monolayers were washed 3 times with ice-cold PBS and fixed in 3.7% formaldehyde. Following permeabilization with 100% methanol for 10 min at −20° C., renal proximal tubular cells monolayers were washed with PBS containing 0.1% BSA and 0.3% Triton X-100 (PBS/0.1% BSA/0.3% Triton X-100) for 15 min at room temperature. Blocking of non-specific binding was performed for 30 min in PBS containing 8% BSA. Following washing with PBS/0.1% BSA/0.3% Triton X-100 for 15 min, renal proximal tubular cells were incubated overnight at 4° C. with the anti-α1 Na⁺-K⁺-ATPase monoclonal antibody (Upstate Biotechnology, Lake Placid, NY) (5 μg/ml) diluted in PBS containing 1% BSA. Monolayers were washed with PBS/0.1% BSA/0.3% Triton X-100 for 30 min and incubated for 3 hr at room temperature with goat anti-mouse IgG fluorescein-conjugated secondary antibody (Chemicon, Temecula, Calif.) diluted in PBS (10 μg/ml). Following washing with PBS/0.1% BSA/0.3% Triton X-100 for 30 min, cells were mounted in mounting media (0.1M Tris-HCl, pH 8.5 containing 0.25% 1,4-diazabicyclooctane, 5% n-propyl gallate, 10% polyvinyl alcohol, and 25% glycerol) and examined using a Zeiss 1 0 confocal laser scanning microscope at a magnification of 800×. Fluorescent images were generated using an argon laser set at 488 nm wavelength and a 520 nm pass barrier filter. Following the establishment of the coordinates of the apical and basal surfaces in renal proximal tubular cells monolayers, 10 optical Z-plane sections were obtained from the basal to apical domain with the step-shift of focal plane of 1 μm. Digital fluorescent images collected from the focal planes were assigned their location relative to the basal or apical surfaces and captured.

### EXAMPLE 8

Statistical Analysis

Data are presented as means ±SE and were analyzed for significance using two-way ANOVA. Multiple means were compared using Student-Newman-Keuls test. Statements of significance were based on P<0.05. Renal proximal tubules isolated from an individual rabbit represented a separate experiment (n=1) consisting of data obtained from 3 culture dishes.

### EXAMPLE 9

Proliferation of Renal Proximal Tubular Cells

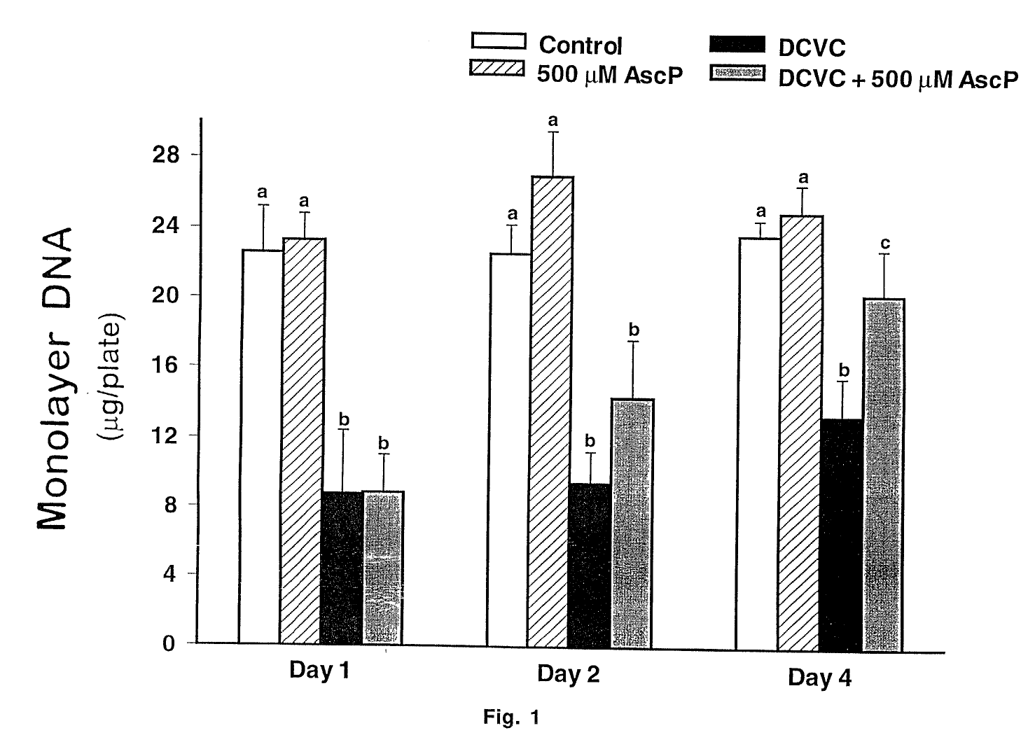

Monolayer DNA contents in control renal proximal tubular cells (RPTC) grown in the presence of 0.05 and 0.5 mM L-ascorbic acid phosphate were equivalent (FIG. 1). Exposure of confluent renal proximal tubular cells to dichlorovinyl-L-cysteine resulted in 61% loss of monolayer DNA at 24 hr following the treatment, regardless of the concentration of L-ascorbic acid phosphate (0.05 mM and 0.5 mM) in the medium during the culture period and the toxicant exposure (FIG. 1). Monolayer DNA contents in dichlorovinyl-L-cysteine-injured renal proximal tubular cells grown in the presence of a physiological concentration (0.05 mM) of AscP did not increase during the recovery period (FIG. 1). In contrast, monolayer DNA contents in dichlorovinyl-L-cysteine-injured renal proximal tubular cells grown in the presence of a pharmacological concentration (0.5 mM) of L-ascorbic acid phosphate increased by 1.6- and 2.3-fold on days 2 and 4, respectively, and was 81% of controls on day 4 (FIG. 1). These data show that dichlorovinyl-L-cysteine-induced cell death and loss are equivalent in renal proximal tubular cells grown in the presence of physiological and pharmacological concentrations of L-ascorbic acid phosphate, but the higher concentration of L-ascorbic acid phosphate promotes renal proximal tubular cells proliferation and regeneration.

### EXAMPLE 10

Mitochondrial Function of Renal Proximal Tubular Cells

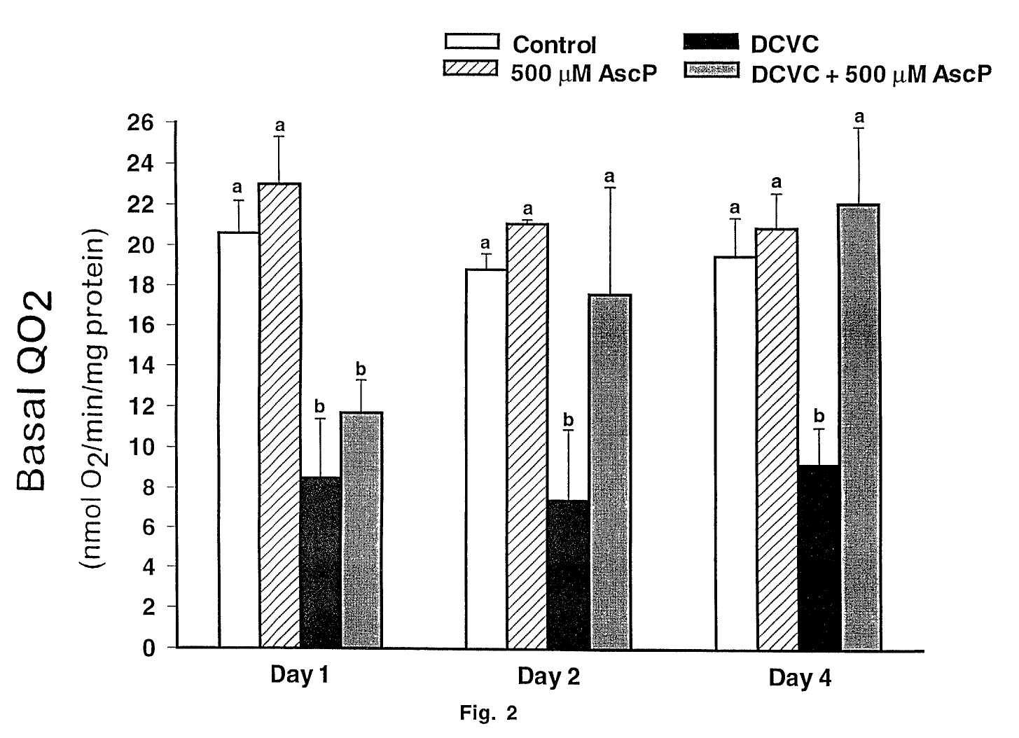

Basal QO₂ was used as a marker of mitochondrial function in renal proximal tubular cells. In control renal proximal tubular cells, basal QO₂ was equivalent in cells grown in the presence of 0.05 and 0.5 mM AscP (FIG. 2). In sublethally injured renal proximal tubular cells grown in the presence of 0.05 mM L-ascorbic acid phosphate, dichlorovinyl-L-cysteine exposure decreased basal QO₂ by 59% at 24 hr following injury. No significant changes in basal QO₂ occurred in these renal proximal tubular cells during the 4 day recovery period (FIG. 2). Likewise, dichlorovinyl-L-cysteine produced a 62% decrease in basal QO₂ in renal proximal tubular cells grown in the presence of 0.5 mM L-ascorbic acid phosphate. However, in contrast to renal proximal tubular cells grown in the presence of a physiological concentration of L-ascorbic acid phosphate, basal QO₂ in renal proximal tubular cells cultured in the presence of a pharmacological concentration of L-ascorbic acid phosphate completely recovered on day 4 following dichlorovinyl-L-cysteine exposure (FIG. 2).

At 24 hr following dichlorovinyl-L-cysteine treatment, ouabain-insensitive QO₂ decreased 32% (9.7±1.6 vs. 6.6±2.6 nmol O₂/min/mg protein in control and dichlorovinyl-L-cysteine-treated renal proximal tubular cells, respectively) in the presence of 0.05 mM L-ascorbic acid phosphate and by 50% (12.9±1.5 vs. 6.5±1.0 nmol O₂/min/mg protein in control and dichlorovinyl-L-cysteine-treated renal proximal tubular cells, respectively) in the presence of 0.5 mM AscP. Ouabain-insensitive QO₂ remained decreased (45%) through day 4 in dichlorovinyl-L-cysteine-injured renal proximal tubular cells grown in the presence of 0.05 mM AscP but fully recovered in injured renal proximal tubular cells cultured in the presence of 0.5 mM L-ascorbic acid phosphate (data not shown). These data show that dichlorovinyl-L-cysteine-induced decreases in the mitochondrial function are equivalent in renal proximal tubular cells grown in the presence of physiological and pharmacological concentrations of L-ascorbic acid phosphate, but a pharmacological concentration of L-ascorbic acid phosphate promotes recovery of this function following sublethal injury.

### EXAMPLE 11

Basolateral Membrane Function of Renal Proximal Tubular Cells

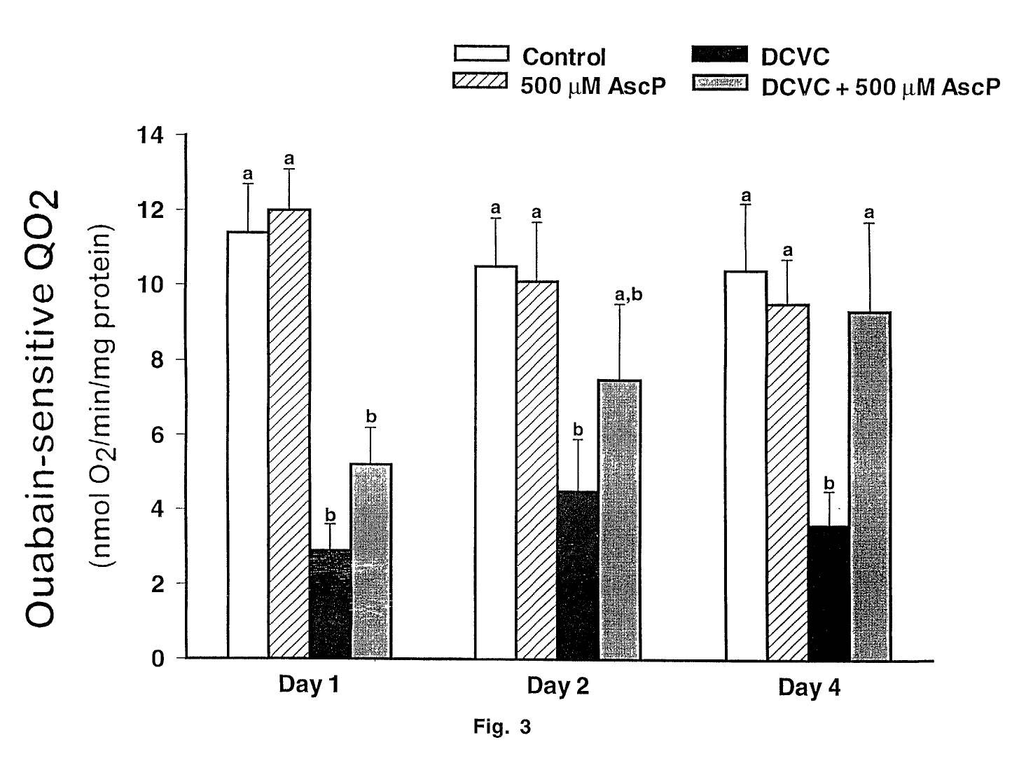

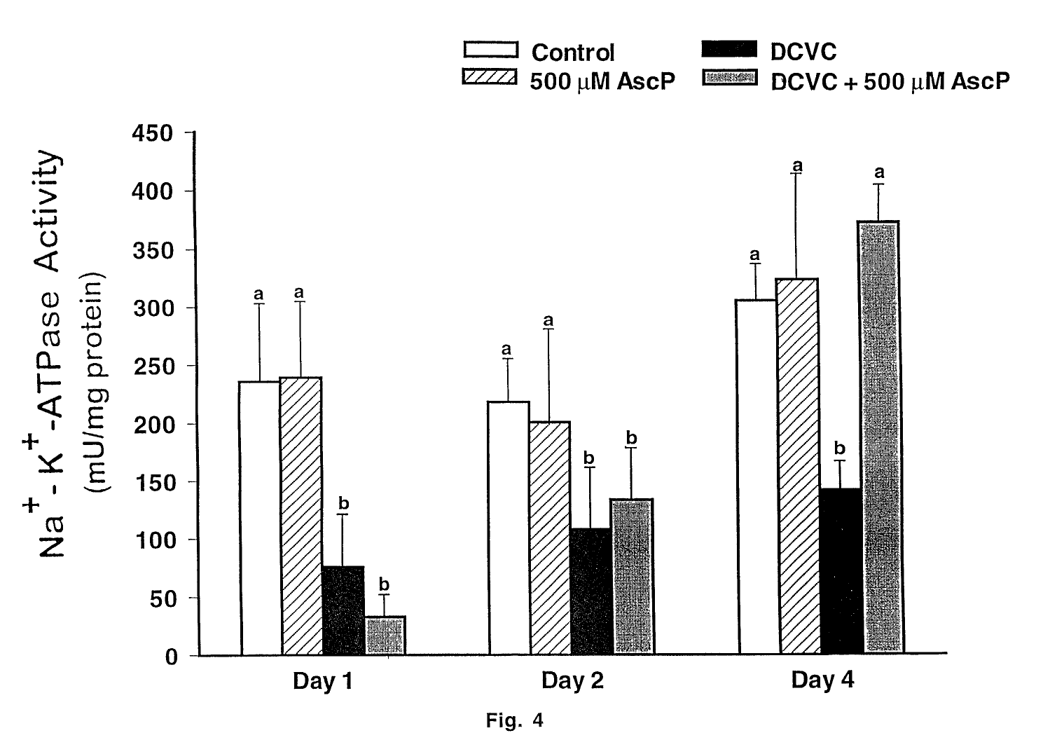

Active Na⁺ transport was used as a marker of basolateral membrane function in renal proximal tubular cells. Active Na⁺ transport in renal proximal tubular cells was assessed by measurements of ouabain-sensitive QO₂ and Na⁺-K⁺-ATPase activity. Ouabain-sensitive QO₂ was equivalent in control renal proximal tubular cells grown in the presence of 0.05 and 0.5 mM L-ascorbic acid phosphate (FIG. 3). At 24 hr following dichlorovinyl-L-cysteine exposure, ouabain-sensitive QO₂ decreased approximately 66% and was not statistically different in renal proximal tubular cells grown in the presence of 0.05 and 0.5 mM L-ascorbic acid phosphate (FIG. 3). Na⁺-K⁺-ATPase activity at 24 hr following dichlorovinyl-L-cysteine treatment was reduced by approximately 77% in cells grown in the presence of 0.05 and 0.5 mM L-ascorbic acid phosphate (FIG. 4). Neither ouabain-sensitive QO₂ nor Na⁺-K⁺-ATPase activity recovered following dichlorovinyl-L-cysteine injury in renal proximal tubular cells grown in the presence of a physiological concentration of L-ascorbic acid phosphate (FIGS. 3 and 4). However, ouabain-sensitive QO₂ and Na⁺-K⁺-ATPase activity recovered following dichlorovinyl-L-cysteine injury in renal proximal tubular cells grown in the presence of 0.5 mM AscP (FIG. 3 and 4). These data show that dichlorovinyl-L-cysteine-induced decreases in active Na⁺ transport and Na⁺-K⁺-ATPase activity are equivalent in renal proximal tubular cells grown in the presence of physiological and pharmacological concentrations of L-ascorbic acid phosphate but a pharmacological concentration of L-ascorbic acid phosphate stimulates repair of these functions following sublethal injury.

### EXAMPLE 12

Subcellular Localization of Na⁺- K⁺-ATPase

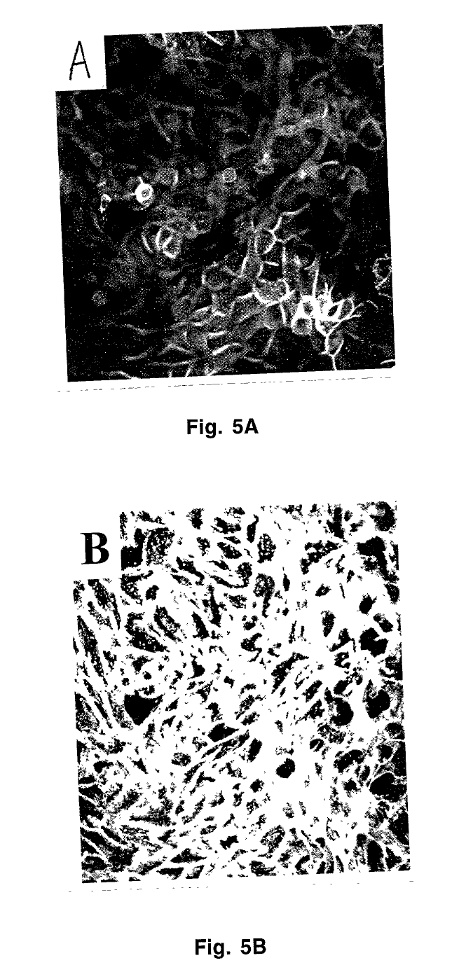

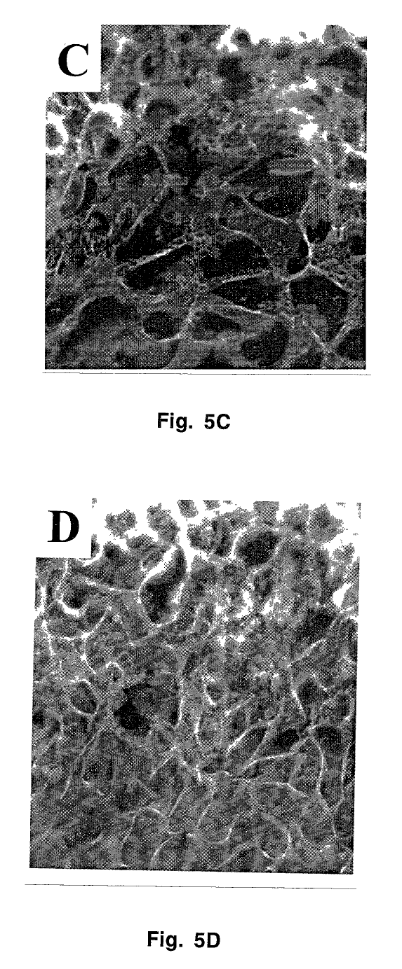

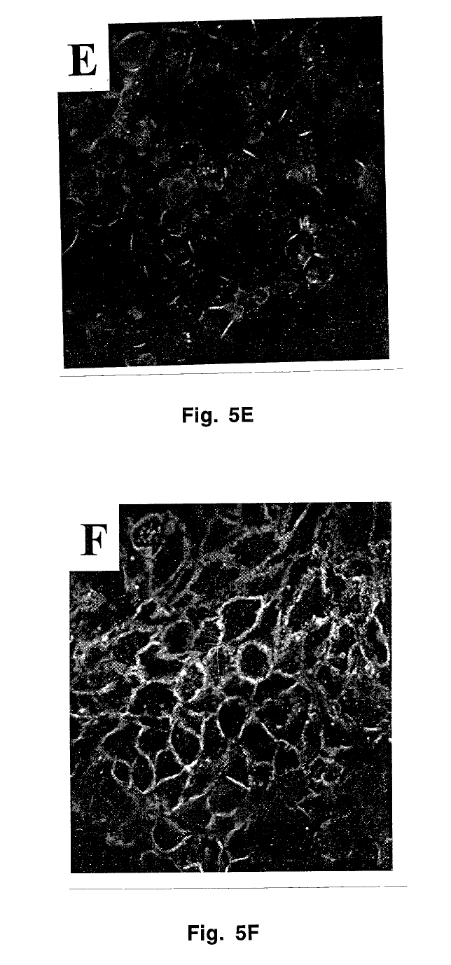

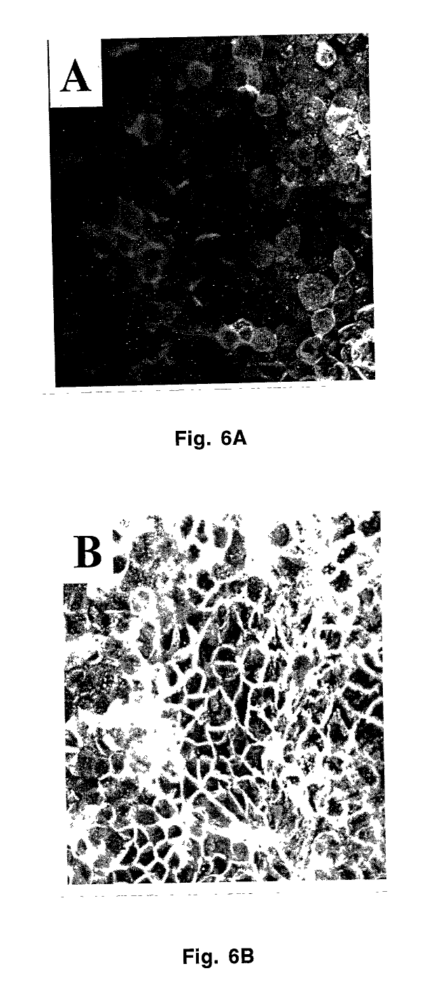

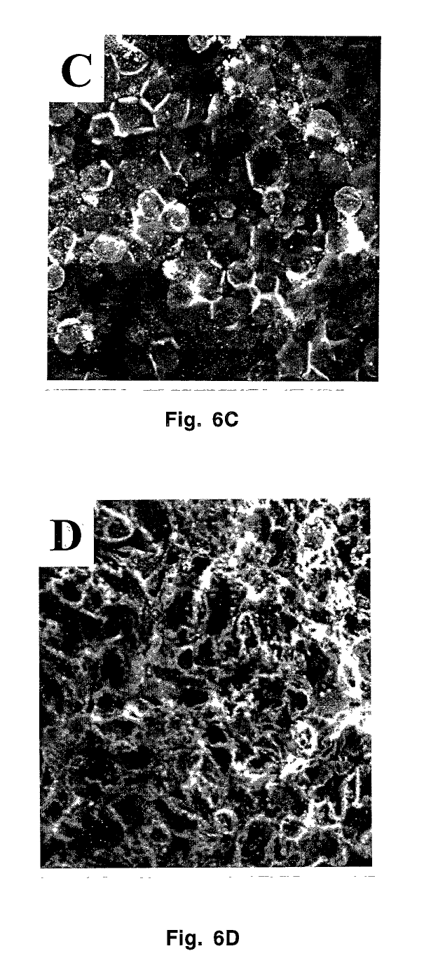

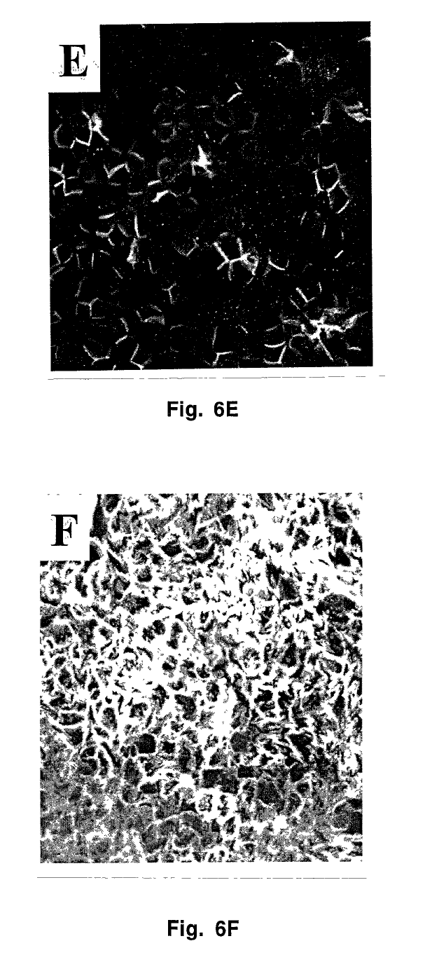

To examine Na⁺-K⁺-ATPase distribution on the plasma membrane of control renal proximal tubular cells, optical Z-plane sections were produced from basal to apical domains and images collected from various focal planes. FIGS. 5 and 6 show sections through apical (A) and basal (B) domains of control renal proximal tubular cells grown in the presence of 0.05 (FIG. 5) and 0.5 mM (FIG. 6) L-ascorbic acid phosphate. While the Na⁺-K⁺-ATPase protein is abundant in the basolateral domain of renal proximal tubular cells, it is almost absent from the apical domain (FIG. 5A and B). These results demonstrate the polarized distribution of Na⁺-K⁺-ATPase on the plasma membrane of confluent renal proximal tubular cell cultures, similar to that found in renal proximal tubular cells in vivo. The data also show that there is no difference in the basolateral Na⁺-K⁺-ATPase protein levels and distribution between renal proximal tubular cells grown in the presence of a physiological (FIG. 5A and B) and a pharmacological (FIG. 6A and B) concentration of L-ascorbic acid phosphate.

DCVC-induced injury was associated with the loss of the Na⁺-K⁺-ATPase protein from the basolateral domain of renal proximal tubular cells independent of the L-ascorbic acid phosphate concentration in the medium (FIG. 5D and 6D). No recovery of the Na⁺-K⁺-ATPase protein occurred in DCVC-injured renal proximal tubular cells grown in the presence of 0.05 mM L-ascorbic acid phosphate (FIG. 5E and F). In contrast, the Na⁺-K⁺-ATPase protein levels of renal proximal tubular cells grown in the presence of 0.5 mM L-ascorbic acid phosphate completely recovered during the 4 day regeneration period following dichlorovinyl-L-cysteine injury. Furthermore, the Na⁺-K⁺-ATPase protein was localized to the basolateral domain in a manner similar to that of controls (FIG. 6E and F).

These data demonstrate that dichlorovinyl-L-cysteine exposure in renal proximal tubular cells induces a loss of the Na⁺-K⁺-ATPase protein from the plasma membrane. The results also show that DCVC-induced loss of Na⁺-K⁺-ATPase from the plasma membrane is equivalent in renal proximal tubular cells grown in the presence of a physiological and pharmacological concentration of L-ascorbic acid phosphate, but a pharmacological concentration of L-ascorbic acid phosphate promotes the restoration of protein levels and polarized distribution of the Na⁺-K⁺-ATPase on the plasma membrane. These observations are consistent with: 1) the lack of recovery of the Na⁺ K⁺-ATPase activity and active Na⁺ transport in DCVC-injured renal proximal tubular cells cultured in the presence of physiological concentrations of L-ascorbic acid phosphate and 2) promotion of recovery of these renal proximal tubular cells functions by pharmacological concentrations of L-ascorbic acid phosphate (FIGS. 3 and 4).

Discussion

Renal dysfunction following toxicant-induced injury may result from cellular injury and decreases in physiological cell functions and also by the inhibition of cellular recovery by certain nephrotoxicants. Recently, it has been demonstrated that renal proximal tubular cells in primary culture undergo complete morphological regeneration of the monolayer following sublethal injury induced by an oxidant (tert-butyl hydroperoxide, TBHP) and that this process is due to cellular repair, proliferation and migration/spreading (Nowak and Schnellmann, 1997; Nowak et al., 1998). The decreases in mitochondrial function, intracellular ATP content, Na⁺-K⁺-ATPase activity, active Na⁺ transport, and Na⁺-coupled glucose uptake in sublethally-injured renal proximal tubular cells after TBHP exposure are followed by complete recovery of these functions, with cellular proliferation and monolayer regeneration preceding the return of mitochondrial and transport functions (Nowak et al., 1998). This recovery is not dependent on exogenous mitogens or factors stimulating cellular repair. Thus, renal proximal tubular cells in primary culture have the autocrine mechanisms necessary for complete morphological and functional repair following sublethal injury induced by an oxidant.

In contrast, dichlorovinyl-L-cysteine exposure that results in a similar degree of cell death and loss (30%) from the monolayer and sublethal injury to the remaining cells, is not followed by monolayer regeneration nor recovery of mitochondrial and transport function (Nowak et al., 1999). The inhibition of renal proximal tubular cells regeneration after dichlorovinyl-L-cysteine-induced injury can be overcome by daily epidermal growth factor (EGF, 10 ng/ml) treatments which suggest, that EGF activates mechanisms of cellular repair that had been inhibited by dichlorovinyl-L-cysteine (Nowak et al., 1999).

Previously, it was demonstrated that ascorbic acid phosphate increases proliferation and mitochondrial and transport functions in renal proximal tubular cells, and promotes morphological regeneration of renal proximal tubular cells following TBHP exposure by stimulation of proliferation and migration/spreading (Nowak and Schnellmann, 1996; Nowak and Schnellmann, 1997). The present study tested the hypothesis that pharmacological concentrations of L-ascorbic acid phosphate promote recovery of renal proximal tubular cells functions following dichlorovinyl-L-cysteine-induced injury. Renal proximal tubular cells were grown in the presence of physiological (0.05 mM) and pharmacological (0.5 mM) concentrations of L-ascorbic acid phosphate and exposed to 0.2 mM dichlorovinyl-L-cysteine to produce cell injury. The results demonstrate that dichlorovinyl-L-cysteine produced a similar degree of cell death and decreases in renal proximal tubular cells functions at both concentrations of L-ascorbic acid phosphate and suggested that pharmacological concentrations of L-ascorbic acid phosphate had no protective effect against dichlorovinyl-L-cysteine-induced injury in renal proximal tubular cells.

In the presence of a physiological concentration of L-ascorbic acid phosphate, the decrease in cell number due to dichlorovinyl-L-cysteine-induced cell death was not followed by proliferation and restoration of the monolayer. These data suggest that dichlorovinyl-L-cysteine exposure inhibits renal proximal tubular cells proliferation. In contrast, proliferation occurred following dichlorovinyl-L-cysteine exposure in renal proximal tubular cells grown in the presence of pharmacological concentrations of L-ascorbic acid phosphate. Previous results suggested that the lack of proliferation following dichlorovinyl-L-cysteine exposure in renal proximal tubular cells grown in the presence of physiological concentrations of L-ascorbic acid phosphate is due to the lack of mitogenic signals in dichlorovinyl-L-cysteine-injured renal proximal tubular cells and that EGF stimulates renal proximal tubular cells proliferation and regeneration following dichlorovinyl-L-cysteine-induced injury (Nowak et al., 1999). The present data show that sublethally-injured renal proximal tubular cells grown in the presence of pharmacological concentrations of L-ascorbic acid phosphate maintain the ability to proliferate and restore the monolayer following dichlorovinyl-L-cysteine-induced injury.

Mitochondrial function, active Na⁺ transport and Na⁺-K⁺-ATPase, and Na⁺-dependent glucose uptake are major targets of dichlorovinyl-L-cysteine in renal proximal tubular cells (Lash and Anders, 1987; Groves et al., 1993; Van de Water et al., 1994, Stevens et al., 1986, Vamvakas et al., 1996, Nowak et al., 1999). In the present model, the decrease in mitochondrial function is observed immediately after dichlorovinyl-L-cysteine removal from the monolayers and prior to any evidence of renal proximal tubular cells injury or death (Nowak et al., 1999). Mitochondrial function in renal proximal tubular cells grown in the presence of physiological concentrations of L-ascorbic acid phosphate did not recover following dichlorovinyl-L-cysteine exposure; in contrast to the complete recovery of this function after oxidant-induced injury (Nowak et al., 1998). However, basal QO₂ recovered on day 2 following dichlorovinyl-L-cysteine exposure in renal proximal tubular cells grown in the presence of pharmacological concentrations of L-ascorbic acid phosphate, demonstrating that that L-ascorbic acid phosphate stimulates the repair of mitochondrial function in renal proximal tubular cells following toxicant injury. Promotion of the recovery of mitochondrial function by pharmacological concentrations of L-ascorbic acid phosphate was not due to protection against dichlorovinyl-L-cysteine toxicity since the decreases in mitochondrial function at 24 hr following dichlorovinyl-L-cysteine exposure were equivalent in the presence of physiological and pharmacological concentrations of L-ascorbic acid phosphate. Therefore, it is concluded that the recovery of mitochondrial function in dichlorovinyl-L-cysteine-injured renal proximal tubular cells grown in the presence of pharmacological concentrations of L-ascorbic acid phosphate is not due to the antioxidant effect of L-ascorbic acid phosphate.

The present results show that active Na⁺ transport is a target of dichlorovinyl-L-cysteine in renal proximal tubular cells and that this function does not recover in renal proximal tubular cells grown in the presence of physiological concentrations of L-ascorbic acid phosphate (FIG. 3). In contrast, pharmacological concentrations of L-ascorbic acid phosphate stimulate recovery of active Na⁺ transport in renal proximal tubular cells following dichlorovinyl-L-cysteine-induced injury. The return of active Na⁺ transport to control levels occurred on day 4 after dichlorovinyl-L-cysteine exposure and followed the recovery of mitochondrial function. The decrease in active Na⁺ transport is the result of the inhibition of Na⁺ K⁺-ATPase activity and loss of Na⁺-K⁺-ATPase protein in dichlorovinyl-L-cysteine-injured renal proximal tubular cells (Nowak et al., 1999). The mechanism of dichlorovinyl-L-cysteine-induced decrease in Na⁺-K⁺-ATPase activity and protein is not clear. Previously, it was shown that dichlorovinyl-L-cysteine causes depolymerization of F-actin and disorganization of cellular cytoskeleton (Van der Water et al., 1994). These alterations are usually associated with loss of cell polarity (Molitoris et al., 1989). Because Na⁺-K⁺-ATPase is localized to basolateral membrane and associated with the cytoskeleton through F-actin, depolymerization of actin contributes to the loss of Na⁺-K⁺-ATPase protein from basolateral membrane of injured cells. Independently, the loss of mitochondrial function and ATP depletion may also contribute to the decrease in Na⁺-K⁺-ATPase activity.

Na⁺-K⁺-ATPase loss after dichlorovinyl-L-cysteine exposure is not followed by the recovery of Na⁺-K⁺-ATPase protein nor its basolateral localization in sublethally-injured renal proximal tubular cells cultured in the presence of physiological concentrations of L-ascorbic acid phosphate. This fact may be due to a deficiency in both F-actin polymerization and repair of actin cytoskeleton, and/or decreased synthesis of new Na⁺-K⁺-ATPase protein. The lack of recovery of mitochondrial function and ATP levels may further arrest the recovery of Na⁺-K⁺-ATPase activity. In contrast, in renal proximal tubular cells cultured in the presence of pharmacological concentrations of L-ascorbic acid phosphate, protein levels of Na⁺-K⁺-ATPase completely recovered following dichlorovinyl-L-cysteine-induced injury. Furthermore, Na⁺-K⁺-ATPase protein in regenerating renal proximal tubular cells was localized mainly to basolateral membrane which indicated the recovery of renal proximal tubular cells plasma membrane polarity. Recovery of Na⁺-K⁺-ATPase protein levels was associated with the return of Na⁺-K⁺-ATPase enzymatic activity and recovery of active Na⁺ transport. In addition, L-ascorbic acid phosphate did not protect against the loss of Na⁺-K⁺-ATPase protein and activity following dichlorovinyl-L-cysteine exposure (FIGS. 4 and 5). Therefore, one can conclude that pharmacological concentrations of L-ascorbic acid phosphate promote recovery of Na⁺-K⁺-ATPase protein and activity through the mechanisms other than the antioxidant effect of this molecule.

The precise mechanism by which the injured epithelium regenerates through proliferation and recovers normal cellular architecture is not known. Ascorbic acid is a well known stimulator of collagen production and deposition into the basement membrane, and previous reports suggested that L-ascorbic acid phosphate may promote cell proliferation and increase cell density through increased collagen deposition (Peterkofsky, 1991; Murad et al., 1983). The composition of the basement membrane may play an essential role in the recovery process of renal proximal tubular cells by providing extracellular signals for proliferative responses and a structural framework for regaining cellular polarity. However, contribution of other potential mechanisms, unrelated to collagen deposition, to the recovery processes in renal proximal tubular cells is possible.

In conclusion, the present results show that: 1) proliferation, mitochondrial function, Na⁺-K⁺-ATPase protein level and activity, and active Na⁺-transport do not recover in dichlorovinyl-L-cysteine-injured renal proximal tubular cells cultured in the presence of physiological concentrations of L-ascorbic acid phosphate, 2) pharmacological concentrations of L-ascorbic acid phosphate promote proliferation and repair of mitochondrial function, recovery of Na⁺-K⁺-ATPase protein level and activity, and return of active Na⁺ transport in dichlorovinyl-L-cysteine-injured RPTC, and 3) stimulation of proliferation and recovery of mitochondrial function and active Na⁺ transport in renal proximal tubular cells by pharmacological concentrations of L-ascorbic acid phosphate is not due to protective effects of L-ascorbic acid phosphate against DCVC-induced cell death and/or decreases in mitochondrial function, Na⁺-K⁺-ATPase activity, and active Na⁺ transport. These data also suggest that the beneficial effects of pharmacological concentrations of ascorbic acid in the kidney are not limited to antioxidant action of this molecule and that ascorbic acid may be an important tool in promoting recovery of renal functions following toxicant-induced injury.

The following references were cited herein:

Alejandro, V. S., W. J. Nelson, P. Huie, R. K. Sibley, D. Dafoe, P. Kuo, J. D. Scandling, and Myers, B. D. (1995). Postischemic injury, delayed function and Na⁺-K⁺-ATPase distribution in the transplanted kidney. Kidney Int. 48, 1308-1315.

Anderson, R. J., and Schrier, R. W. (1997). Acute renal failure. In: Diseases of the Kidney, edited by R. W. Schrier, and C. W. Gottschalk. Boston, Mass.: Little Brown, 1997 p. 1069-1113.

Chen, Q., T. W. Jones, and Stevens, J. L. (1994). Early cellular events couple covalent binding of reactive metabolites to cell killing by nephrotoxic cysteine conjugates. J. Cell. Phys. 161, 293-302.

Chi, W. M., I. K. Berezesky, M. W. Smith, and Trump, B. F. (1995). Changes in [Ca²⁺]ᵢ in cultured rat proximal tubular epithelium: an in vitro model for renal ischemia. Biochim. Biophys. Acta 1243, 513-520.

Cuppage, F. E., and Tate, A. (1967). Repair of the nephron following injury with mercuric chloride. Am. J. Pathol. 51, 405-429.

Dekant, W., Vamvakas, S., and Anders, M. W. (1994). Formation and fate of nephrotoxic and cytotoxic glutathione S-conjugates: Cysteine conjugate β-lyase pathway. Adv. Pharmacol. 27, 115-162.

Elfarra, A. A., Jackson, I., and Anders, M. W. (1986). Mechanisms of S-(1,2-dichlorovinyl) glutathione-induced nephrotoxicity. Biochem. Pharmacol. 35, 283-288.

Glaumann, B., Glaumann, H., Berezesky, I. K., and Trump, B. F. (1977). Studies on cellular recovery from injury. II. Ultrastructural studies on the recovery of the pars convoluta of the proximal tubule of the rate kidney from temporary ischemia. Virchows Archiv B. Cell Pathology. 24, 1-18.

Goldstein, R. G., and Schnellmann, R. G. (1995). Toxic responses of the kidney. In: Cassarett & Doull's Toxicology, C.D. Klaassen, ed., McGraw-Hill, New York, pp. 417-442.

Groves, C. E., Lock, E. A., and Schnellmann, R. G. (1991). Role of lipid peroxidation in renal proximal tubule cell death induced by haloalkene cysteine conjugates. Toxicol. Appl. Pharmacol. 10, 54-62.

Groves, C. E., Hayden, P. J., Lock, E. A. and Schnellmann, R. G. (1993). Differential cellular effects in the toxicity of haloalkene and haloalkane cysteine conjugates to rabbit renal proximal tubules. J. Biochem. Tox. 8, 49-56.

Ilinskaja, O., and Vamvakas, S. (1996). Alterations of the renal function in the isolated perfused rat kidney system after in vivo and in vitro application of S-(1,2-dichlorovinyl)-L-cysteine and S-(2,2-dichlorovinyl)-L-cysteine. Arch. Toxicol. 70, 224-229.

Labarca, C., and Paigen, K. (1980). A simple, rapid, and sensitive DNA assay procedure. Anal. Biochem. 102, 344-352.

Lash, L. H. (1994). Role of metabolism in chemically induced nephrotoxicity. In: Mechanisms of Injury and Renal Disease and Toxicity, ed. R. S. Goldstein. Boca Raton, Fla.: CRC Press, pp. 207-237.

Lash, et al., (1987). Mechanism of S-(1,2-dichlorovinyl)-L-cysteineand S-(1,2-dichlorovinyl)-L-homocysteine-induced renal mitochondrial toxicity. Mol. Pharm. 32, 549-556.

Lowry, et al., (1951). Protein measurement with the Folin phenol reagent. J. Biol. Chem. 193, 265-275.

Kribben, et al., (1994). Evidence for role of cytosolic free calcium in hypoxia-induced proximal tubule injury. J. Clin. Invest. 93, 1922-1929. McCoy, C. E., Selvaggio, A. M., Alexander, E. A., and Schwartz, J. H. (1988). Adenosine triphosphate depletion induces a rise in cytosolic free calcium in canine renal epithelial cells. J. Clin. Invest. 82, 1326-1332.

Meister, A., Tate, S. S., and Griffith, O. W. (1989). Reduced proximal tubule Na⁺ transport following ischemic injury. J. Membr. Biol. 107, 119-127.

Mohrmann, M., Pauli, A., Ritzer, M., Schonfeld, B., Seifert, B., and Brandis, M. (1992) Inhibition of sodium-dependent transport systems in LLC-PK1 cells by metabolites of ifosfamide. Renal Physiol. Biochem. 15, 289-301.

Molck, A. M., and Friis, C. (1997). The cytotoxic effect of paraquat to isolated renal proximal tubular segments from rabbits. Toxicology. 122, 123-32.

Molitoris, B. A., Chan, L. K., Shapiro, J. I., Conger, J. D., and Falk, S. A. (1989). Loss of epithelial polarity: a novel hypothesis for reduced proximal tubule Na⁺ transport following ischemic injury. J. Membr. Biol. 107, 119-127.

Molitoris, B. (1991). Ischemia-induced loss of epithelial polarity: Potential role of the actin cytoskeleton. Am. J. Physiol. 29, F769-778.

Molitoris, B. A., Dahl, R., and Geerdes, A. (1992). Cytoskeleton disruption and apical redistribution of proximal tubule Na⁺-K⁺-ATPase during ischemia. Am. J. Physiol. 263, F488-F495.

Monteil, C. Leclere, C. Fillastre, J. P., and Morin J. P. (1993). Characterization of gentamicin-induced dysfunctions in vitro: the use of optimized primary cultures of rabbit proximal tubule cells. Renal Failure. 15, 475-483.

Moore, R. B., and Green, T. (1988). The synthesis of nephrotoxin conjugates of glutatione and cysteine. Toxicol. Environ. Chem. 17, 153-162.

Murad, S., Tajima, S., Johnson, G. R., Sivarajah, A., and Pinnal, S. R. (1983). Collagen synthesis in cultured human skin fibroblasts: effect of ascorbic acid and its analogs. J. Invest. Dermatol. 81, 158-162.

Nowak, G., and Schnellmann, R. G. (1995). Integrative effects of EGF on metabolism and proliferation in renal proximal tubular cells. Am. J. Physiol. 26, C1317-C1325.

Nowak, G., and Schnellmann, R. G. (1996). L-ascorbic acid regulates growth and metabolism of renal cells: improvements in cell culture. Am. J. Physiol. 271, C2072-C2080.

Nowak, G., and Schnellmann, R. G. (1997). Renal cell regeneration following oxidant exposure: inhibition by TGF-β₁ and stimulation by ascorbic acid. Toxicol. Appl. Pharmacol. 145, 175-183.

Nowak, G., Aleo, M. D., Morgan, J. A., and Schnellmann, R. G. (1998). Recovery of cellular functions following oxidant injury. Am. J. Physiol. 274, F509-F515.

Nowak, G. Keasler, K. B., McKeller, D. E., and Schnellmann, R. G. (1999). Differential effects of EGF on repair of cellular functions after dichlorovinyl-L-cysteine-induced injury. Am. J. Physiol. 276, F228-F236.

Peterkofsky, B. (1991). Ascorbate requirement for hydroxylation and secretion of procollagen: relationship to inhibition of collagen synthesis in scurvy. Am. J. Clin. Nutr. 54, 1135S-1140S.

Spiegel, D. M., Wilson, P. D., and Molitoris, B. A. (1989). Epithelial polarity following ischemia: a requirement for normal cell function. Am. J. Physiol. 256, F430-F436.

Stevens, J. L., Hayden, P., and Taylor, G. (1986). The role of glutathione conjugate metabolism and cysteine conjugate beta-lyase in the mechanism of S-cysteine conjugate toxicity in LLC-PK1 cells. J. Biol. Chem. 261, 3325-3332.

Toback, F. G. (1992). Regeneration after acute tubular necrosis. Kidney Int. 41, 226-246.

Toback, F. G., Kartha, S., and Walsh-Reitz M. M. (1993). Regeneration of kidney tubular epithelial cells. Clin. Invest. 71, 861-866.

Vamvakas, S., Richter, H., and Bittner, D. (1996). Induction of dedifferentiated clones of LLC-PK1 cells upon long-term exposure to dichlorovinylcysteine. Toxicology. 106, 65-74.

Van de Water, et al., (1994). In vivo and in vitro detachment of proximal tubular cells and F-actin damage: consequences for renal function. Amer. J. Phys. 267, F888-899.

Venkatachalam, et al., (1981). Mechanism of proximal tubule brush border loss and regeneration following mild renal ischemia. Lab. Invest. 45, 355-365.

Weinberg, et al., (1997). Cytosolic-free calcium increases to greater than 100 micromolar in ATP-depleted proximal tubules. J. Clin. Invest. 100, 713-722.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended a s limitations on the scope of the invention. Changes therein and other uses will occur to those skilled in the art which are encompassed within the spirit of the invention as defined by the scope of the claims.

What is claimed is:

1. A method of recovering cellular functions in cells following injury, comprising the step of:
contacting said cells with ascorbic acid or a salt of ascorbic acid.

2. The method of claim 1, wherein said cellular function is elected from the group consisting of proliferation, mitochondrial function, Na⁺-K⁺-ATPase protein expression, Na⁺-K⁺-ATPase protein activity, and active Na⁺ transport.

3. The method of claim 1, wherein said ascorbic acid is L-ascorbic acid phosphate.

4. The method of claim 1, wherein the concentration of said ascorbic acid is from about 0.05 mM to about 0.5 mM.

5. An ophthalmic composition comprising a therapeutically effective amount of ascorbic acid or a salt of ascorbic acid and an ophthalmically acceptable carrier.

6. The composition of claim 5, wherein the composition is in a form selected from the group consisting of aqueous eye drops, liposomes, microspheres, proteins, collagen and soft contact lenses.

7. A pharmaceutical composition in the form of an ointment, lotion, cream or spray, comprising a therapeutically effective amount of ascorbic acid or a salt of ascorbic acid and a topically acceptable carrier.

8. A pharmaceutical composition, comprising a therapeutically effective amount of ascorbic acid or a salt of ascorbic acid on a solid support, wherein said solid support comprises a dressing.

9. The pharmaceutical composition of claim 8, wherein said solid support comprises an adsorbent material.

10. The pharmaceutical composition of claim 9, wherein said adsorbent material is attached to an adhesive strip.

11. A method of recovering cellular functions following injury in an individual in need of such treatment, comprising the step of:
administering a therapeutically effective amount of ascorbic acid or a salt of ascorbic acid to said individual.

12. The method of claim 11, wherein said injury is selected from the group consisting of halogenated hydrocarbons-induced nephrotoxicity, ischemia- and drug-induced acute renal failure, and glomerulonephritis, and skin abrasions, cuts, and burns.

13. The method of claim 12, wherein said halogenated hydrocarbons is dichlorovinyl-L-cysteine.

14. The method of claim 12, wherein said cellular function is selected from the group consisting of proliferation, mitochondrial function, Na⁺-K⁺-ATPase protein expression, Na⁺-K⁺-ATPase protein activity, and active Na⁺ transport.

15. A method of recovering cellular functions following injury to the eye of an individual in need of such treatment, comprising the step of:
administering the ophthalmic composition of claim 5 to said individual.

16. The method of claim 15, wherein said injury is selected from the group consisting of acute injury to the eye, eye diseases associated with the over production of collagen (conjunctivitis, diabetes mellitus), eye disease associated with the under production of collagen (alkali burns, rheumatoid arthritis).

17. A product for delivery of a therapeutically effective amount of ascorbic acid comprising:
(A) a strip comprising:
(i) a flexible substrate sheet; and
(ii) a therapeutically effective amount of ascorbic acid deposited onto said substrate sheet.
